(19) United States
(12) Patent Application Publication
(10) Pub. No.: US 2013/0066351 A1
(43) Pub. Date: Mar. 14, 2013

(54) APPARATUS FOR CREATING AN ANNULAR INCISION IN SOFT TISSUE

(71) Applicants: Christopher Kenneth Giardina, Chapel Hill, NC (US); Shane Anthony Saunders, Roswell, GA (US); Khaled Nizar Kashian, Suwanee, GA (US); Rebeca Kyker Bowden, Seattle, WA (US); Jorge Alejandro Baro, Madison, WI (US); Kanitha Devi Kim, Atlanta, GA (US)

(72) Inventors: Christopher Kenneth Giardina, Chapel Hill, NC (US); Shane Anthony Saunders, Roswell, GA (US); Khaled Nizar Kashian, Suwanee, GA (US); Rebeca Kyker Bowden, Seattle, WA (US); Jorge Alejandro Baro, Madison, WI (US); Kanitha Devi Kim, Atlanta, GA (US)

(21) Appl. No.: 13/415,035

(22) Filed: Mar. 8, 2012

Related U.S. Application Data
(60) Provisional application No. 61/450,389, filed on Mar. 8, 2011.

(51) Int. Cl. A61B 17/32

(52) U.S. Cl. 606/180

(57) ABSTRACT

The present invention relates to a device and method for cutting an annular piece out of the soft tissue of a selected mammal. The device can be utilized in surgical situations such as capsularhexis. The device comprises one or more blades which when rotated, cut an annular path.

This application claims priority of U.S. provisional application No. 61/450,389 filed on Mar. 8, 2011 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the surgical cutting of soft tissue. More particularly, the present invention relates to surgical devices and procedures that may require surgeons to perform annular cuts with precision in terms of position, size, and circularity.

2. Description of Related Art

One procedure in which it is useful to make an annular incision is capsulorhexis, an early step in cataract removal surgery. When performing capsulorhexis, an incision is made in the cornea, a tool inserted into the eye through the incision, and a circular incision made in the eye's lens capsule in order to gain access to the cataract. Although various devices have been created to perform this function, there remains a need for a device that can perform annular incisions with precision in terms of position, size and circularity.

### BRIEF SUMMARY OF THE INVENTION

In one aspect, a surgical device is provided for creating an annular incision in a soft tissue. The surgical device includes a cutting head comprising at least one blade and in one embodiment a pair of diametrically-opposed cutting blades, and an actuator that induces mechanical motion of the cutting head such that the cutting blades follow an annular path about an axis of rotation.

In another aspect, a method is provided for performing surgery on a soft tissue. The method includes actuating a cutting head to induce mechanical motion of the cutting head such that one or more cutting blades follow an annular path about an axis of rotation. In some embodiments, the method further includes inserting the cutting head through an incision made in first a soft tissue before actuating the cutting head on a separate soft tissue.

In one embodiment, the invention relates to a surgical device for creating an annular incision in the soft tissue of a mammal in a surgical procedure comprising:
a) a cutting head comprising at least one cutting blade having a face and at least one axial arm attached to each blade, each arm having a distal end attached to the blade and a proximal end, the proximal end of each arm joining at an axial rotation point; wherein the blades are curved and positioned such that when the arms are rotated circumferentially about the axial rotation point, the blades trace an annular cutting path; and
b) an actuator attached to the cutting head that causes the cutting head to rotate circumferentially about the axial rotation point.

In another embodiment the invention relates to a method of performing a surgical procedure on a first soft tissue of a mammal comprising cutting an annular piece of the first soft tissue the method comprising:
a) selecting a surgical device comprising:
   i. a cutting head comprising at least one cutting blade having a face and at least one axial arm attached to each blade, each arm having a distal end attached to the blade and a proximal end, the proximal end of each arm joining at an axial rotation point; wherein the blades are curved and positioned such that when the arms are rotated circumferentially about the axial rotation point, the blades trace an annular cutting path;
   ii. an actuator attached to the cutting head that causes the cutting head to rotate circumferentially about the axial rotation point,
b) positioning the cutting blades against the first soft tissue in a position to cut the annular piece of tissue; and
c) engaging the actuator to cut the tissue.

### BRIEF DESCRIPTION OF THE DRAWINGS

FIG. 1 is a perspective view of the blade assembly on a cutting head of a surgical instrument constructed in accordance with one or more embodiments of the present invention with a rectangular face.

FIG. 2 is a perspective view of the front portion of a surgical instrument constructed in accordance with one or more embodiments of the present application.

FIG. 3 is a perspective view of the surgical instrument of FIG. 1 and FIG. 2.

FIG. 4 is a perspective view of a broad blade shaped blade face.

FIG. 5 is a perspective view of a triangular blade shaped blade face.

FIGS. 6a-6c are side views of the surgical procedure of the invention.

FIG. 7 is a perspective view of a circular blade of the invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “surgical device” is a device designed for creating an annular incision in the soft tissue of a mammal (such as a human or a non-human animal) in a surgical procedure. The surgical device comprises a “cutting head” which is a device supporting one or more blades that together trace an annular cutting path. There can be a singular curved blade or a completely circular blade. One or more “axial arms” support each blade as shown in the drawings. The arms meet at an axial rotation point for spinning the blades in an annular manner. The actuator is attached to the cutting head such that it causes the cutting head to rotate circumferentially about the axial rotation point such that the blades trace an annular path.

Surgical devices and procedures are provided in the present invention for making annular incisions, for example, in soft biological tissues of a mammal. In an exemplary embodiment, the surgical device may be utilized in a cataract removal surgery, for example, when performing capsulorhexis by first insertion of a device through an incision made in the cornea. In other embodiments, the surgical device may be employed to make annular incisions in other types of soft biological tissue. Advantageously, in some embodiments, the cutting head of the surgical device may fit through a small incision (such as about a 3-5 mm long incision or less or in one embodiment about 3-10 mm or less) in a second outer tissue, be actuated to produce an annular incision in a first inner tissue, and then be removed from the tissue site through the incision in the outer tissue.

A surgical device is provided for creating an annular incision in a soft tissue. In one embodiment the surgical device may include a cutting head comprising a pair of diametrically-opposed cutting blades or a circular blade, and an actuator that induces mechanical motion of the cutting head such that the diametrically-opposed cutting blades follow an annular path about an axis of rotation.

In certain embodiments, each of the cutting blades comprises a cutting edge that is arcuate in shape. For example, each of the cutting blades may have a cutting edge that projects toward a cutting surface in the shape of an arc. In other embodiments, the cutting edge projects toward the cutting surface in the form of a narrow cutting tip. The cutting edge may comprise any material suitable for cutting including, but not limited to, metal (such as stainless steel), diamond or materials such as flexible metals such as nitinol.

In some embodiments, each of the diametrically-opposed cutting blades are attached to at least one axial arm. For example, the cutting blades may be attached to a common connecting arm which acts as an axial arm for two blades. The axial arm may be angularly rotated by a drive shaft extending coaxially with the axis of rotation of the cutting head.

In some embodiments, the actuator comprises a crank arm operatively coupled to the cutting blades. For example, the crank arm may be attached to a drive shaft that extends coaxially with the axis of rotation of the cutting blades such that the crank arm and drive shaft rotate in unison. In some embodiments, the actuator may further include a linear shaft operatively coupled to the crank arm such that linear movement of the linear shaft induces the cutting blades to follow the annular path. Such an actuating mechanism advantageously may overcome strength limitations of miniaturized gear systems. In other embodiments, the actuator may include a worm gear that drives a drive gear on the drive shaft. In other embodiments, the actuator may include a pulley drive system. For example, a continuous flexible cable may wrap around and drive a pulley or gear that is coupled to the drive shaft.

In some embodiments, the actuator configured to induce an oscillation of the diametrically-opposed cutting blades along the annular path. For example, movement of the actuator may cause the diametrically-opposed cutting blades to alternately travel clockwise and counterclockwise directions along the annular path. Alternatively, the actuator may be configured to induce motion of the diametrically-opposed cutting blades in a single angular direction along the annular path.

In some embodiments, the cutting head is configured to be insertable through about a 3-5 or less mm long incision, in one embodiment about 3-10 mm incision. For example, the surgical device may include a hand piece and a wand extending between the hand piece and the cutting head. The cutting head may be positionable in an inserting configuration in which the cutting blades are aligned with the wand. Thus, when inserted through the incision, with two blades, one cutting blade passes through the incision before the other cutting blade. In some embodiments the cutting head is foldable or flexible to fit the incision or just sized to fit the incision. In other embodiments, the surgical device may be utilized to cut tissue without inserting the cutting head through an incision.

In some embodiments, the hand piece further comprises a thumb slide operatively connected to the actuator such that motion of the thumb slide is translated into motion of the actuator. The thumb slide may be selectively positionable in a first position in which the cutting head is positionable in an inserting configuration in which the diametrically-opposed cutting blades are aligned with the wand. The thumb slide may also be selectively positionable in a second position such that movement of the thumb slide between the first and second position induces motion of the cutting head. In another embodiment the blades are motorized and rotate/oscillate the blades based on activation of an electric power motor.

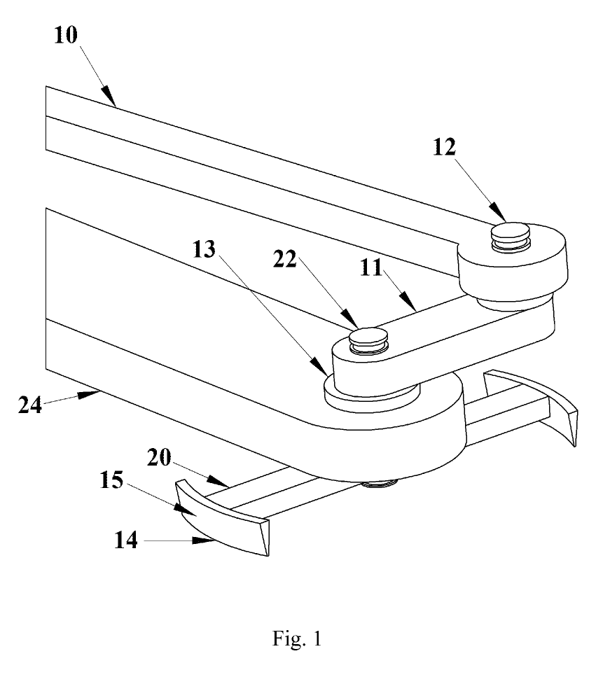

FIG. 1 is a perspective view of an embodiment of a cutting head 25 for a surgical device. In the illustrated embodiment, each cutting blade 14 is arcuate in shape and has a rectangular face 15. The cutting blades 14 are attached at opposite ends of an axial arm 20. The axial arm 20 is coupled to a drive shaft 22 which passes through the wand 24. The drive shaft 22 at the axial rotation point is connected to a crank arm 11 which is separated from the wand 24 with a bearing 13. The crank arm 11 is connected to a linear shaft 10 by a pivot joint 12.

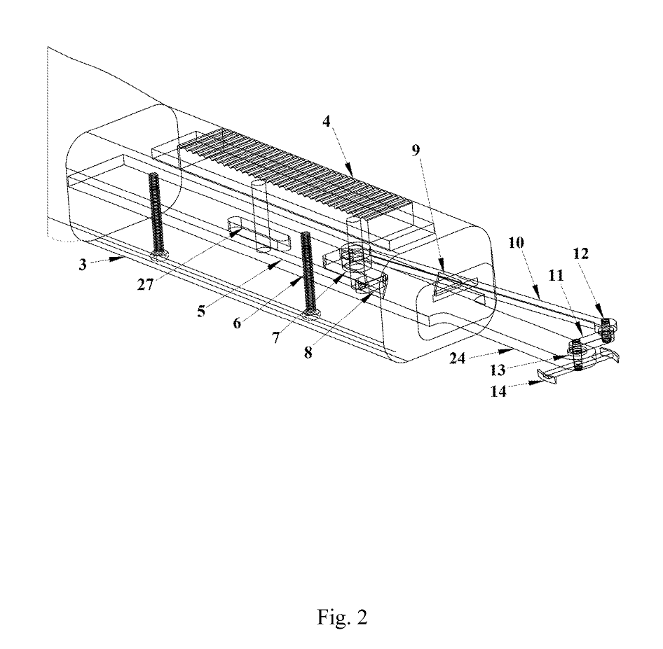

As illustrated in FIG. 2, there is a perspective view of the surgical instrument. The cutting head 25 is attached at the end of a wand 24 that extends from a hand piece. The hand piece includes a housing 3 which houses at least a portion of the actuator. The housing 3 also houses a guide plate 5 which guides the movement of the linear shaft 10. The guide plate 5 may be secured within the housing 3 by one or more fasteners 6. A thumb slide 4 is located on the outside of the housing 3 and is connected within the housing 3 to the linear shaft 10. The movement of the thumb slide 4 and the linear shaft 10 may therefore be constrained by the shape of a slot 27 provided in the guide plate 5. A bearing 7 may be positioned between the thumb slide 4 and the guide plate 5 to allow the thumb slide 4 to move freely along the guide plate 5 through the slot 27. In some embodiments, one or more bumpers 8 and/or a spring 9 may be employed to ensure that torque is applied to the cutting head in the desired direction when the linear shaft 10 and crank 11 are aligned in parallel.

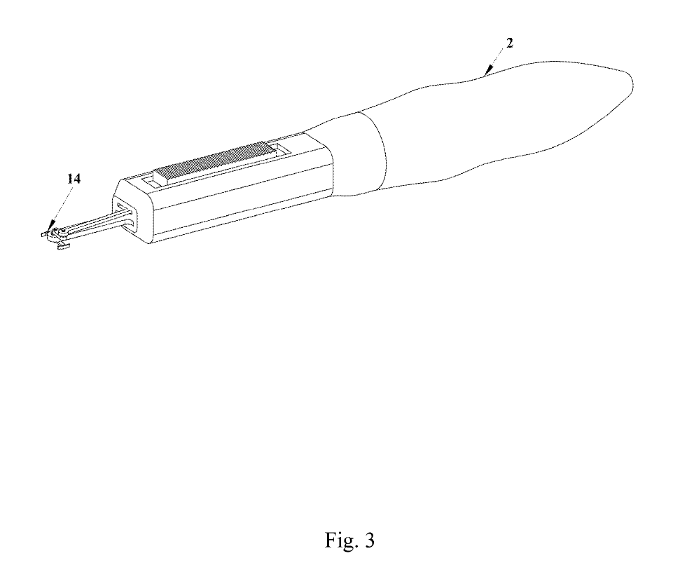

As illustrated in FIG. 3, the hand piece also includes a handle 2. The handle 2 may comprise a slip-resistant material. For example, the handle 2 may comprise a rubber or elastic material and/or may include tactile or gripping features. The housing may be made of any durable material, for example, ABS thermoplastic. The cutting blades 14, wand, connecting rod, linear shaft, crank arm, and thumb slide (as shown in FIG. 2) may be made of stainless steel or other appropriate material.

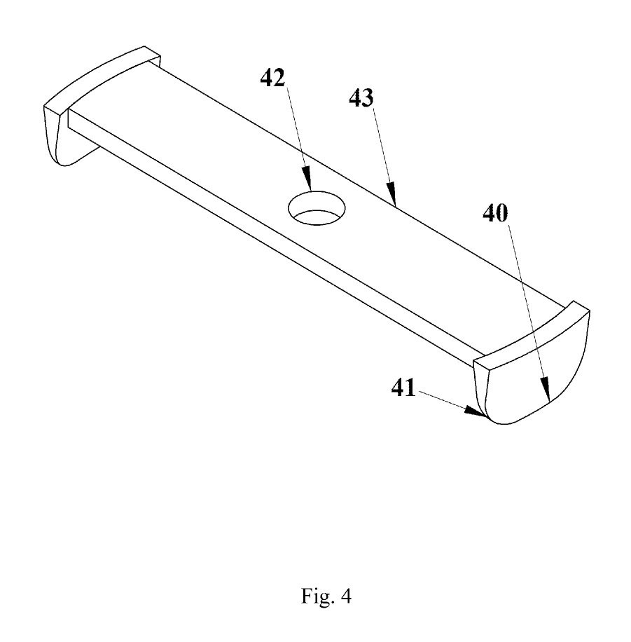

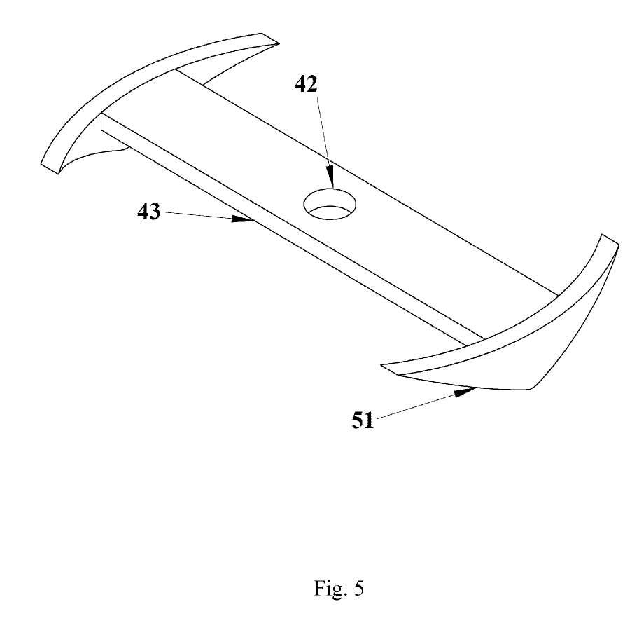

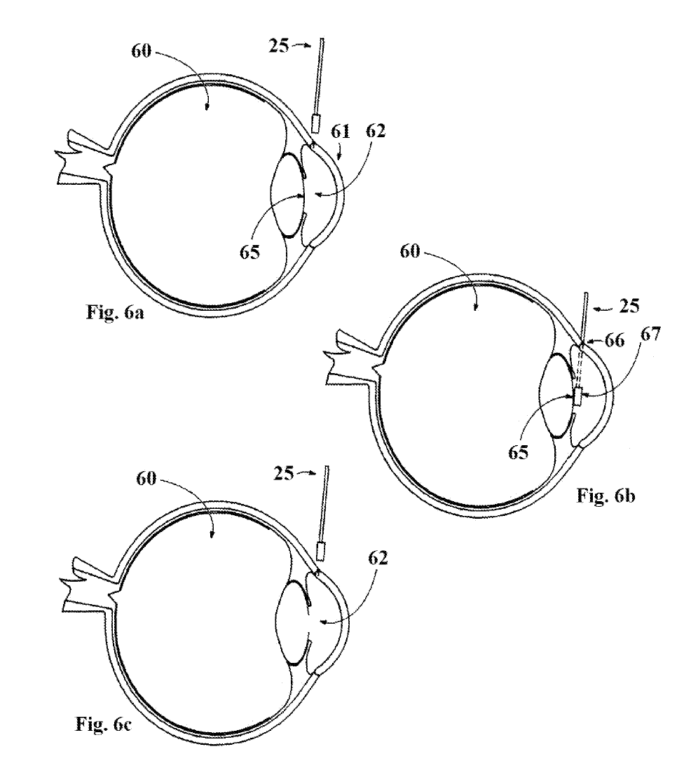

The surgical devices disclosed herein may be employed in various surgical applications. FIG. 4 is a perspective view of a set of dual blades 40 and 41 with a broad face 42 and arms 43. FIG. 5 is a perspective of a triangular blade face 51. For example, the surgical devices disclosed herein may be employed to produce annular incisions in various soft biological tissues including, but not limited to, ophthalmic tissue, cardiac tissue, uterine tissue, arteriovenous tissue, and dermal tissue. In an exemplary embodiment, the surgical device may be employed in a cataract removal procedure. For example, the surgical device may be employed when performing capsulorhexis. FIGS. 6a, 6b, and 6c depict respectively device 25 approaching eye 60 ready to be inserted into cornea 61 to reach the aqueous humor 62 to reach lens 65. In FIG. 6b device 25 is inserted into incision 66 and blades 67 are placed against the capsule of the lens 65 and the tissue cut. FIG. 6c depicts the removed tissue and device 25 removed from the eye 60 at the end of the surgery.

When employed in a cataract removal procedure, the surgical device may be employed after an initial incision is made in the cornea. In some embodiments, the incision in the cornea may be relatively short, for example, about 3 mm. In other embodiments as large as about 10 mm. The cutting head of the surgical device may then be inserted through the incision and placed over the lens capsule. The cutting head may be placed at the desired location by aligning the drive shaft with the center of the region to be cut. The user may then actuate the thumb slide to make an annular incision. For example, the user may advance the thumb slide all the way forward, and then return the thumb slide to the original position. Once the thumb slide is returned to the original position, the annular incision may be complete and the cutting head may be removed from the tissue site through the corneal incision.

In the embodiment illustrated in FIG. 2, movement of the thumb slide 4 will cause the linear shaft 10 to move in the same direction as the thumb slide 4. The motion of the linear shaft 10 causes the crank arm 11 to rotate in a circular motion which will ultimately rotate the drive shaft and the cutting blades 14 attached thereto. In the illustrated embodiment, the bumpers 8 and spring 9, which is attached to the linear shaft 10, exert a force in a lateral direction relative to the direction of travel of the linear shaft at points of zero lever torque, ensuring continuous motion.

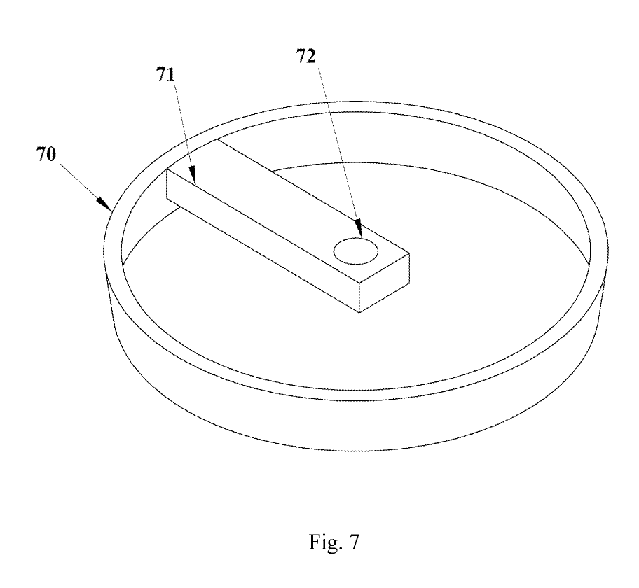

In FIG. 7 there is shown a circular blade 70 having axial arm 71 and axial rotation point 72.

It should be apparent that the foregoing relates only to some embodiments of the present invention and that numerous changes and modifications may be made herein without departing from the spirit and the scope of the invention as defined by the following claims and equivalents thereof.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A surgical device for creating an annular incision in the soft tissue of a mammal in a surgical procedure comprising:
a) a cutting head comprising at least one cutting blade having a face and at least one axial arm attached to each blade, each arm having a distal end attached to the blade and a proximal end, the proximal end of each arm joining at an axial rotation point; wherein the blades are curved and positioned such that when the arms are rotated circumferentially about the axial rotation point, the blades trace an annular cutting path; and
b) an actuator attached to the cutting head that causes the cutting head to rotate circumferentially about the axial rotation point.

2. The surgical device according to claim 1 wherein there are two diametrically opposed cutting blades.

3. The surgical device according to claim 1 where in there is one circular blade.

4. The surgical device according to claim 1 wherein the cutting head is foldable into a configuration that makes a portion of its profile smaller for insertion into an incision smaller than the cutting head in a non-folded configuration.

5. The surgical device according to claims 1 through 4 wherein the cutting head is configured for insertion into a tissue incision of about 3 to 10 mm or less.

6. The surgical device according to claim 1 wherein the blade has at least one side edge that is sharpened.

7. The surgical device according to claim 1 wherein the blade face is rectangular.

8. The surgical device according to claim 1 wherein the blade face is triangular.

9. The surgical device according to claim 1 wherein the blade face has a broad blade shape.

10. The surgical device according to claim 1 wherein the actuator is crank arm operatively coupled to the axial rotation point to rotate the blades circumferentially about the axial rotation point.

11. The surgical device according to claim 9 wherein the actuator comprises a linear shaft that is operatively coupled to the crank arm such that linear movement of the linear shaft induces the blades to rotate circumferentially about the axial rotation point.

12. The surgical device according to claim 1 wherein the actuator is configured to oscillativly rotate the blades.

13. The surgical device according to claim 1 wherein the actuator is configured to rotate the blades circumferentially in a single direction.

14. The surgical device according to claim 1 wherein the actuator comprises a thumb slide operatively connected to the actuator such that motion of the thumb slide causes the blades to rotate circumferentially about the axial rotation point.

15. A method of performing a surgical procedure on a first soft tissue of a mammal comprising cutting an annular piece of the first soft tissue the method comprising:
a) selecting a surgical device comprising:
i. a cutting head comprising at least one cutting blade having a face and at least one axial arm attached to each blade, each arm having a distal end attached to the blade and a proximal end, the proximal end of each arm joining at an axial rotation point; wherein the blades are curved and positioned such that when the arms are rotated circumferentially about the axial rotation point, the blades trace an annular cutting path;
ii. an actuator attached to the cutting head that causes the cutting head to rotate circumferentially about the axial rotation point;
b) positioning the cutting blades against the first soft tissue in a position to cut the annular piece of tissue; and
c) engaging the actuator to cut the tissue.

16. The surgical procedure according to claim 15 wherein the cutting head is inserted through a second soft tissue of the mammal in order to position the cutting blades against the first soft tissue.

17. The surgical procedure according to claim 16 wherein the procedure is capsulorhexis.

18. The surgical procedure according to claim 16 wherein the second soft tissue is the cornea of an eye.
